(19) United States
(12) Patent Application Publication
(10) Pub. No.: US 2003/0100883 A1
(43) Pub. Date: May 29, 2003

(54) Cartridge for liquid insulin

(76) Inventors: Lars Thougaard Kristensen, Vekso (DK); Steffen Hansen, Hillerod (DK); Lars Peter Klitmose, Gentofte (DE)

Correspondence Address: Reza Green, Esq., Novo Nordisk of North America, Inc., Suite 6400, 405 Lexington Avenue, New York, NY, 10174-6401, US

(21) Appl. No.: 10/223,739

(22) Filed: Aug. 23, 2002

Related U.S. Application Data
(60) Provisional application No. 60/317,593, filed on Sep. 7, 2001.

(30) Foreign Application Priority Data
Aug. 31, 2001 (DK) PA 2001 01282

(51) Int. Cl. A61B 19/00

(52) U.S. Cl. 604/411; 604/203; 604/232; 604/415

(57) ABSTRACT

A glass cartridge which can be utilized both in an insulin pump system and in an insulin injection system. The glass cartridge contains U200 insulin. In order to obtain a suitable accuracy of the doses delivered by the system, the inside diameter of the cartridge must be in the range 7.45 to 9.32 mm.

### CROSS REFERENCE TO RELATED APPLICATIONS

This application claims the benefit of priority under 35 U.S.C. §119 of U.S. Provisional Application No. 60/317,593, filed on Sep. 7, 2001 and Danish Application PA 2001 01282, filed on Aug. 31, 2001; the contents of both are hereby incorporated by reference in their entirety.

### BACKGROUND OF THE INVENTION

1. Field of the Invention

The invention relates to ampoules or cartridges for insulin delivery systems. Such cartridges are commonly shaped as a glass tube being at one end closed by a piston, which may be pressed into the tube to expel the content of the tube at the other end of the tube. This other end is formed as a bottleneck, the outer end of which may be pierced by an injection needle or a catheter through which the content is expelled. 2. RELATED ART

Glass cartridges are widely known for various medicament delivery systems. They are especially used for insulin delivery systems, and are usually supplied pre-filled with either 1.5 ml of insulin or 3.0 ml of insulin. A 1.5 ml cartridge usually has an inside diameter around 6.85 mm and a 3.0 ml cartridge usually has an inside diameter around 9.25 mm. These known cartridges are pre-filled with insulin having a concentration on 100 International Units (IU) pr. ml. A 1.5 ml cartridge therefore contains 150 IU and a 3.0 ml cartridge contains 300 IU.

The typical diabetes patient will require a certain amount of insulin either injected or infused into their body every day. Some patients need as much as 100 IU per day, in which case the 3.0 ml cartridge is recommended. The patient loads the cartridge into either an injection system or a pump system and injects or infuses the insulin into their body at a prescribed rate, either through an injection needle or through a catheter inserted into their body. Once the cartridge is empty it is disposed of and a new cartridge is loaded into the delivery system.

Glass is the most preferred material for cartridges containing insulin, since glass are both chemically and biologically inert so that insulin can be stored within the glass cartridge without reactions occurring between the liquid insulin and the glass material. Glass has the additional advantage that it can be thermally sterilized. Glass cartridges are produced from long glass tubes, which are cut up into smaller tubes, one end of which is melted so that a small opening remains. The opposite open end of the tubes is provided with a movable piston, which are usually manufactured from rubber or plastic.

Cartridges made from glass however has the disadvantage that the inside diameter is variable due to the manufacturing process. The inside diameter of glass cartridges, varies by 0.1 mm at an average inside diameter of about 10 mm. However, finer tolerances are available by pre sorting the glass cartridges or by tightened monitoring of the glass process. The following table shows the tolerances typically available today:

| | | | | | | | |
|---|---|---|---|---|---|---|---|
| Inside diameter | 7 | 7.5 | 8 | 9.25 | 10 | 11 | 12 |
| Tolerances +/− | 0.05 | 0.05 | 0.06 | 0.06 | 0.08 | 0.09 | 0.1 |

These tolerances of the cartridge are a major problem for the dose accuracy of the injected or infused insulin. The dose accuracy of an insulin delivery system is the subject of the ISO standard 11608-1. This standard prescribes that a dose in the range 0 to 20 IU most have an accuracy of +/−1 IU, i.e. a nominal dose of 20 IU most contain between 19 to 21 IU. The ISO standard allows a tolerance of doses smaller than 20 IU to be within +/−1 IU, while the tolerances of a dose exceeding 20 IU most be within +/−5%. The most difficult part of the standard to meet is therefore typically the demand for accuracy on +/−1 IU for a dose of 20 IU.

A large diameter combined with a large tolerance provides large variations in the cross section area of the glass cartridge, which will result in large tolerances in the volume delivered by the insulin delivery system. This is however not a major problem when the insulin has a concentration on only 100 IU per ml.

### SUMMARY OF THE INVENTION

It is a constant aim for manufactures of insulin delivery systems to minimize their systems. A delivery system however always has to include a cartridge. Manufactures therefore have a great demand for smaller and more compact cartridges. Never the less no one wants to compromise the number of International Units contained in the cartridge. One way of solving this Gordian knot is by increasing the concentration of the insulin contained in the cartridge. By increasing the concentration up to 200 IU pr ml, a 1.5 ml cartridge is able of containing 300 IU.

With the before mentioned large variations in the cross section area of the glass cartridges it will apparently be increasingly difficult to meet the ISO 11608-1 standard when using a liquid U200 insulin.

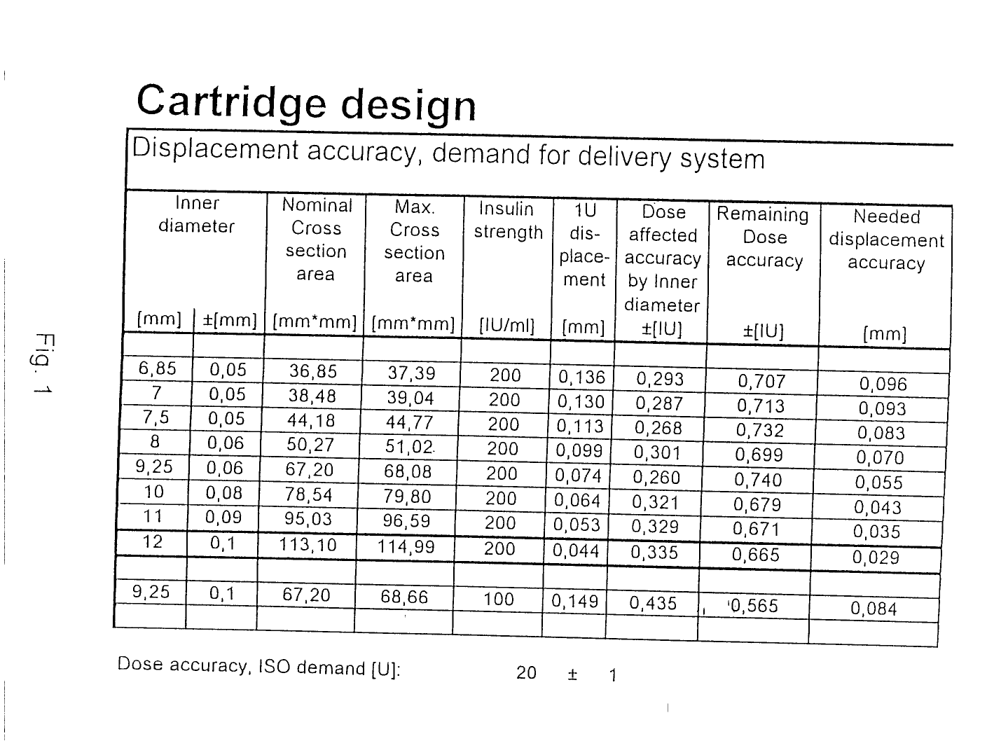

In the table in FIG. 1, the displacement accuracy is calculated for different dimensions of glass cartridges. The two first columns show the different dimensions and tolerances of the inner diameter of various cartridges. The nominal and maximal cross section areas of the cartridges are calculated in column 3 and 4. Column 5 recites the insulin concentration.

The distance the front wall of the piston most be moved forward inside the cartridge in order to expel 1 IU of insulin is calculated in column 6. This movement is calculated on basis of the nominal diameter using the following formula:

$$H = \frac{V}{π \cdot {R}^{2}}$$

V: Volume

R: Radius

H: Displacement

One IU of liquid U200 insulin has the volume of 0.005 ml, the front wall of the piston in e.g. a cartridge having a nominal inside diameter of 9.25 mm most therefore be displaced by 0.074 mm in order to expel one IU.

Column 7 indicates how much the tolerances in the cross section area influence the dose accuracy. It can be seen that for a cartridge with a nominal diameter of 9.25 mm, +/−0.260 IU of the tolerance on +/−1 IU given in the ISO standard are consumed by the tolerance of the cross section area of the cartridge. The tolerance is calculated by extracting the minimum cross section area from the maximum cross section area shown in column 4, and multiplying this difference with the one unit displacement shown in column 6 and with the insulin concentration. The remaining +/−0.740 IU shown in column 8 are then available for the imprecision of the insulin delivery system, including the slack of interface to the cartridge.

The remaining part of the tolerance listed in column 8, is in column 9 expressed in millimeters by multiplying the displacement needed for expelling 1 IU with the remaining tolerance available for the imprecision of the insulin delivery system.

Once again reciting the numerals for a cartridge having a nominal inside diameter of 9.25 mm, the front wall of the piston most be moved a distance equal to 20 times 0.074 mm i.e. 1.488 mm+/−0.055 mm (from 1.433 to 1.543 mm) in order to deliver a dose of 20 IU within a tolerance of +/−1 IU.

The volume expelled within these tolerances can be calculated using the following formula:

V(IU)=CS·D·IC

V(IU): Volume expressed in IU

CS: Cross section area of the cartridge (=π·R²)

D: Displacement of piston

IC: Insulin concentration

The following table show the expelled volume measured in International Units for a cartridge having a nominal diameter of 9.25 mm and tolerances of +/−0.06 mm, i.e. an inside diameter between 9.19 mm and 9.31 mm, equaling a cross section area of the volume of the cartridge between 66.33 mm² and 68.08 mm², when the cartridge is used in an insulin delivery system which can move the piston forward with a tolerance of +/−0.055 mm. The forward movement hence being in the range 1.433 mm to 1.543 mm.

| | 1.433 mm | 1.488 mm | 1.543 mm |
|---|---|---|---|
| 66.33 mm² | 19.01 IU | 19.74 IU | 20.47 IU |
| 67.20 mm² | 19.26 IU | 19.99 IU | 20.74 IU |
| 68.08 mm² | 19.51 IU | 20.26 IU | 21.00 IU |

The mechanical insulin delivery systems available today all have quit a large imprecision due to the mechanical system. The leading injection devices has a displacement accuracy around +/−0.083, meaning that the front wall of the piston can only be moved forward within tolerances of approximately +/−0.083 mm. When using U200 insulin the maximum allowable inner diameter of the cartridge must, according to the table shown in FIG. 1, be smaller than a nominal 7.5 mm cartridge in order to meet the demands set up in the ISO 11608-1 standard. Cartridges having an inside diameter larger than 7.5 mm all needs a displacement accuracy smaller than +/−0.083 mm in order to meet the ISO standard. They are therefore not suitable for use in ordinary insulin pen systems.

Insulin pumps for pump treatment of diabetes usually have a more precise mechanism than mechanical injection devices due to the presence of a motor mechanism, which is also the case for motor driven injection devices. It is however not necessary with a precise mechanism in insulin pumps due to the presence of a continuous insulin delivery profile.

In recent years mechanical precision injection devices has been developed which has a higher degree of accuracy than the known injection devices. In fact these new injection devices are able of moving the piston forward within tolerances of approximately +/−0.055 mm.

It has therefore shown that cartridges containing liquid U200 insulin and having a diameter from the lower tolerance limit of a nominal 7.5 mm cartridge i.e. 7.45 mm to the upper tolerance limit of a nominal 9.25 mm cartridge i.e. 9.31 can be used both in pumps and in precision injection devices having tolerances within +/−0.055 mm to +/−0.083 mm, without dispensing from the requirements of ISO 11608-1.

These requirements are fulfilled with a cartridge comprising a distal end and a proximal end connected by a cylindrical wall forming a vessel containing liquid insulin, the distal end being provided with a flange closed by a flexible membrane sealingly secured against the flange, and the proximal end being closed by a piston which can be moved into said cartridge which accommodates the liquid insulin in the variable space between the flexible membrane and a front wall of the piston, wherein, the liquid insulin is an U200 insulin, and that the cylindrical wall has an inside diameter in the range 7.45 mm to 9.31 mm, such that said cartridge can be utilized both in an insulin pump system and in an insulin injection system.

A glass cartridge having an inside diameter between 7.45 and 9.31 mm will, when filled with a liquid U200 insulin, be able to fulfil the ISO 11608-1 standard when the cartridge is being used in a precision insulin delivery system having a displacement accuracy of the mechanism advancing the piston within the range from 0.055 mm to 0.083 mm.

Glass cartridges with an inside diameter in the specified ranges can therefore be used both in insulin pump systems and in insulin injection systems.

When operating in the lower range of the inside diameters specified it is ensured that, the cartridge can be made very slim and with very narrow tolerances, leaving maximum tolerances for the imprecision of the insulin delivery system.

A glass cartridge having a nominal inside diameter of 7.5 mm needs a length of the stroke zone of approximately 34 mm in order to contain a volume of approximately 1.5 ml. When containing 1.5 ml of a liquid U200 insulin, the total amount of International Unit is 300 IU, which for most patient will be sufficient for three days of treatment. When using the cartridge in a pump system, the pump is usually connected to the body of the user through a catheter. Due to inflammation of the skin at the site where the catheter is inserted, it is normally recommended to change the catheter and the site approximately every third day. A cartridge containing insulin for minimum three days are therefore to be preferred.

Due to the size of the connecting zone and the piston zone, the overall length of such a cartridge will be approximately 52 mm.

When operating in the upper range of the inside diameters specified it is ensured that the cartridge can be made very short and still leave sufficient tolerances for the imprecision of the insulin delivery system. A glass cartridge having a nominal inside diameter of 9.25 mm needs a length of the stroke zone of approximately 23 mm in order to contain a volume of approximately 1.5 ml, resulting in an overall length of approximately 44 mm.

### BRIEF DESCRIPTION OF THE DRAWINGS

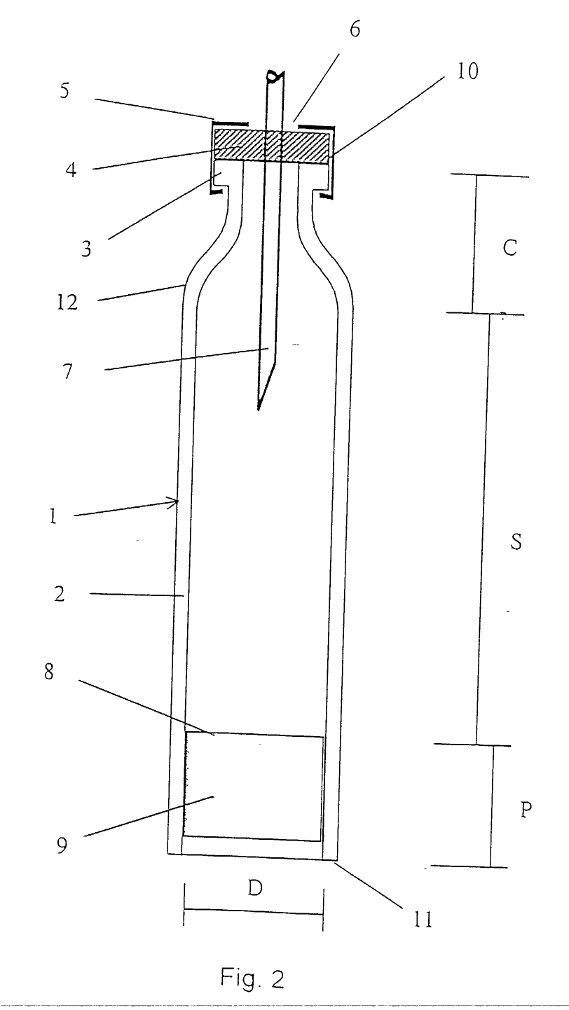

The invention will be explained more fully below in connection with a preferred embodiment and with reference to the drawings in which: FIG. 1 Shows a table of the displacement accuracy for different cartridge designs FIG. 2 Shows a glass cartridge including needle penetration according to the invention.

The figures are schematic and simplified for clarity, and they just show details, which are essential to the understanding of the invention, while other details are left out. Throughout, the same reference numerals are used for identical or corresponding parts.

### DETAILED DESCRIPTION OF THE INVENTION

A table of the displacement accuracy for different cartridge designs is shown in FIG. 1. Glass cartridges containing a liquid U200 insulin and having a nominal diameter between 7.5 mm and 9.25 mm leaves room for a displacement accuracy between +/−0.083 and +/−0.055 mm, which is needed if the cartridge shall be used both for injection devices and for pump systems and fulfil the ISO 11608-1 standard. It can also be seen from the table in FIG. 1 that the displacement accuracy of an insulin delivery system using a cartridge having a nominal diameter of 9.25 mm must be 0.084 mm when the cartridge contains a liquid U100 insulin.

Referring to FIG. 2 it may be convenient to define that, the term “distal end” of the cartridge 1 is meant to refer to the end carrying the conduit 7 through which the insulin is expelled, whereas the term “proximal end” is meant to refer to the opposite end carrying the piston 9.

A cartridge 1 comprising a cylindrical wall 2 is disclosed in FIG. 2. The cylindrical wall 2 is at the distal end 10 of the cartridge terminated in a neck part ending in a circumferential flange 3 against which a piercable and flexible membrane 4 is held sealingly by a metal cap 5. At a central part of the membrane 4 the metal cap 5 has an opening 6 through which the membrane 4 is exposed. A hollow conduit 7, such as an injection needle or a catheter can be stuck through the membrane 4 to communicate with the inner space of the cartridge 1 in which the liquid insulin is stored between the membrane 4 and a front wall 8 of a piston 9 which fits into the cartridge 1.

The piston 9 is usually made from a suitable rubber material, such that it is tightly sealed against the inside of the cylindrical wall 2. The inside diameter of the glass cartridge is indicated with D in FIG. 1.

The cartridge 1 is divided into three different zones. The first zone is the connecting zone C, which extends from the distal end 10 of the cartridge 1 to the shoulder 12. Due to the reduced diameter of the cylindrical wall 2 of the cartridge 1 on the part of the cylindrical wall 2 lying between the distal end 10 of the cartridge 1 and the shoulder 12, the piston 9 cannot be moved beyond the shoulder 12 and into the neck part area of the cartridge 1. The insulin contained in the neck part of the cartridge 1 can therefore not be pressed out of the cartridge, and will hence be disposed of when the cartridge 1 is discarded.

The second zone is the stroke zone S, which extends from the shoulder 12 to the front wall 8 of the piston 9. Only the insulin contained in the stroke zone can be utilized for injection or infusion.

The third zone is the piston zone P, which extends from the proximal end 11 of the cartridge 1 to the front wall 8 of the piston 9. This piston zone P holds the piston 9 and is therefore not available for the insulin contained in the cartridge 1.

The liquid insulin captured between the front wall 8 of the piston 9 and the flexible membrane 4 and within the inside diameter D of the cylindrical wall 2 will be pressed out through the hollow conduit 7, which at the not shown other end is inserted into the person in need for insulin, when the piston 9 is moved forward inside the cartridge 1.

Some preferred embodiments have been shown in the foregoing, but it should be stressed that the invention is not limited to these, but may be embodied in other ways within the subject matter defined in the following claims.

We claim:

1. A glass cartridge for a precision insulin delivery system, said cartridge comprising a distal end and a proximal end connected by a cylindrical wall forming a vessel containing liquid insulin, the distal end being provided with a flange-closed by a flexible membrane sealingly secured against the flange, and the proximal end being closed by a piston which can be moved into said cartridge which accommodates the liquid insulin in the variable space between the flexible membrane and a front wall of the piston,
wherein,
the liquid insulin is an U200 insulin, and that the cylindrical wall has an inside diameter in the range 7.45 mm to 9.31 mm, such that said cartridge can be utilized both in an insulin pump system and in an insulin injection system.

2. A glass cartridge for a precision insulin delivery system according to claim 1, wherein the inside diameter of said cartridge is in range 7.45 to 7.55 mm.

3. A glass cartridge for a precision insulin delivery system according to claim 2, wherein said cartridge has three zones: a connecting zone (C), a stroke zone (S) and a piston zone (P), the stroke zone (S) having a length of approximately 34 mm, such that the stroke zone (S) has a volume of approximately 1.5 ml.

4. A glass cartridge for a precision insulin delivery system according to claim 3, wherein the total length of said cartridge is approximately 52 mm.

5. A glass cartridge for a precision insulin delivery system according to claim 1, wherein the inside diameter of said cartridge is in range 9.19 to 9.31 mm.

6. A glass cartridge for a precision insulin delivery system according to claim 5, whereinsaid cartridge has three zones: a connecting zone (C), a stroke zone (S) and a piston zone (P), the stroke zone (S) having a length of approximately 23 mm, such that the stroke zone (S) has a volume of approximately 1.5 ml.

7. A glass cartridge for a precision insulin delivery system according to claim 6, wherein the total length of said cartridge is approximately 44 mm.
